(19) United States
(12) Patent Application Publication
(10) Pub. No.: US 2005/0138739 A1
(43) Pub. Date: Jun. 30, 2005

(54) Use of polymers for preserving the color in colored hair

(75) Inventors: Torsten Henning, Bad Soden (DE); Matthias Loffler, Niedernhausen (DE); Markus Meder, Werbach (DE)

Correspondence Address: CLARIANT CORPORATION;INTELLECTUAL PROPERTY DEPARTMENT, 4000 MONROE ROAD, CHARLOTTE, NC, 28205, US

(73) Assignee: Clariant GmbH

(21) Appl. No.: 10/977,404

(22) Filed: Oct. 29, 2004

(30) Foreign Application Priority Data
Nov. 4, 2003 (DE) 10351352.3
Dec. 2, 2003 (DE) 10356255.9

(51) Int. Cl. A61K 7/13

(52) U.S. Cl. 8/405

(57) ABSTRACT

The invention relates to the use of one or more polymers chosen from polyethylene glycols, ethoxylated quaternary ammonium compounds and oligoesters, in particular in cosmetic formulations, for stabilizing the color in colored keratin fibers, in particular in human hair, and for protecting these keratin fibers against color loss.

The invention relates to the use of polymers for stabilizing the color in colored keratin fibers, particularly human hair, and for protecting against color loss.

Colored hair suffers from loss of color during washing. Some cosmetic products attempt to prevent this and to protect the color.

EP 509 922 describes the use of polydiorganosiloxane functionalized as thiol.

WO 94/07455 describes the use of an active ingredient combination of alkoxylated wool wax alcohols with 1 to 50 molecules of ethylene oxide per alcohol molecule and pyrrolidonecarboxylic acid and/or derivative.

DE 197 35 865 describes the use of an aqueous-based composition comprising at least one quaternary ammonium compound, green tea extract and a cationic polymer.

EP 940 404 describes the use of at least one organosiloxane which optionally contains at least one group which acts as antioxidant.

U.S. Pat. No. 6,143,286 describes a method of color stabilization using a composition which comprises one or more hair-treatment active ingredients and a siloxane with difunctional and trifunctional units.

However, the color protection shampoos and hair after-treatment compositions with color protection function in the marketplace are hitherto unsatisfactory. After several hair washes, the hair color is often lost despite using products which claim color protection. In addition, known active ingredients with color protection are often not soluble in water and thus often lead to unstable formulations.

It was therefore the object to provide substances which are able to preserve the color in colored keratin fibers, in particular human hair, or to effect protection of colored keratin fibers against color loss, and which do not have the abovementioned disadvantages.

Surprisingly, it has now been found that said object is achieved by the following polymer:
A) polyethylene glycols of the formula H(OCH₂CH₂)ₙOH where n is 150 to 900,
B) ethoxylated quaternary ammonium compound of the formula 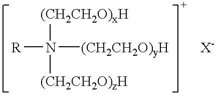
   in which R is C₄- to C₃₀-alkyl, x, y and z, in each case independently of one another, are a number from 1 to 300, and X⁻ is an anionic counterion, and
C) oligoesters which are obtained by condensation of one or more dicarboxylic acids or esters thereof and one or more polyhydric alcohols, optionally in the presence of addition products of an alkylene oxide onto alcohols, alkylphenols or alkylamines, preferably in the presence of methyl polyethylene glycol.

The present invention therefore provides the use of one or more polymers chosen from
A) polyethylene glycols of the formula (1)
   H(OCH₂CH₂)ₙOH (1),
   in which n is an integer between 150 and 900,
B) ethoxylated quaternary ammonium compounds of the formula (2) 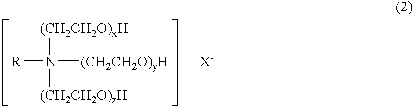
   in which
R is C₄- to C₃₀-alkyl,
x, y and z, in each case independently of one another, are a number from 1 to 300, and
X⁻ is an anionic counterion, and
C) oligoesters which are obtained by condensation of one or more dicarboxylic acids or esters thereof and one or more polyhydric alcohols, optionally in the presence of addition products of an alkylene oxide onto alcohols, alkylphenols or alkylamines, preferably in the presence of methyl polyethylene glycol, for preserving the color in colored keratin fibers, preferably human hair.

Surprisingly, it has also been found that a combination of polymers of two or all three described types of polymer is able to preserve the color in colored keratin fibers, particularly human hair, in a synergistically particularly good way.

When using the polymers to preserve the color in colored keratin fibers, in a preferred embodiment of the invention, one or more polyethylene glycols of the formula (1) as in component A) and one or more ethoxylated quaternary ammonium compounds of the formula (2) as in component B) are used.

When using the polymers to preserve the color in colored keratin fibers, in a further preferred embodiment of the invention, one or more polyethylene glycols of the formula (1) as in component A) and one or more oligoesters as in component C) are used.

When using the polymers to preserve the color in colored keratin fibers, in a further embodiment of the invention, one or more ethoxylated quaternary ammonium compounds of the formula (2) as in component B) and one or more oligoesters as in component C) are used.

When using the polymers to preserve the color in colored keratin fibers, in a further embodiment of the invention, one or more polyethylene glycols of the formula (1) as in component A), one or more ethoxylated quaternary ammonium compounds of the formula (2) as in component B) and one or more oligoesters as in component C) are used.

Preferably, the polymers for the use according to the invention are present in a cosmetic formulation.

The invention also further provides cosmetic formulations comprising at least two polymers chosen from
A) polyethylene glycols of the formula (1)
   H(OCH₂CH₂)ₙOH (1),
   in which n is an integer between 150 and 900,
B) ethoxylated quaternary ammonium compounds of the formula (2) 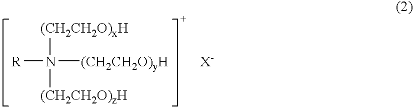
   in which
R is C₄- to C₃₀-alkyl,
x, y and z, in each case independently of one another, are a number from 1 to 300, and
X⁻ is an anionic counterion, and
C) oligoesters which are obtained by condensation of one or more dicarboxylic acids or esters thereof and one or more polyhydric alcohols, optionally in the presence of addition products of an alkylene oxide onto alcohols, alkyl phenols or alkylamines, preferably in the presence of methyl polyethylene glycol.

In a preferred embodiment of the invention, the cosmetic formulations comprise one or more polyethylene glycols of the formula (1) as in component A) and one or more ethoxylated quaternary ammonium compounds of the formula (2) as in component B).

In a further preferred embodiment of the invention, the cosmetic formulations comprise one or more polyethylene glycols of the formula (1) as in component A) and one or more oligoesters as in component C).

In a further preferred embodiment of the invention, the cosmetic formulations comprise one or more ethoxylated quaternary ammonium compounds of the formula (2) as in component B) and one or more oligoesters as in component C).

In a further preferred embodiment of the invention, the cosmetic formulations comprise one or more polyethylene glycols of the formula (1) as in component A), one or more ethoxylated quaternary ammonium compounds of the formula (2) as in component B) and one or more oligoesters as in component C).

In the compounds of the formula (1), n is preferably an integer between 220 and 800.

In the compounds of the formula (2), R is preferably C₈- to C₂₂-alkyl.

In a preferred embodiment of the invention, x, y and z, in each case independently of one another, are a number from 1 to 20, in particular a number from 1 to 4.

In the compounds of the formula (2), the sum x+y+z is a number from 3 to 900. In a further preferred embodiment of the invention, the sum x+y+z is a number from 4 to 50, in particular a number from 4 to 10.

Accordingly, in a particularly preferred embodiment of the invention, R in formula (2) is C₈-C₂₂-alkyl and the sum x+y+z is a number from 4 to 50, preferably a number from 4 to 10.

In the compounds of the formula (2), the anionic counterion X⁻ is preferably chosen from halide anions, OH⁻ and deprotonated weak acids, preferably deprotonated lactic acid.

In a preferred embodiment-of the invention, the oligoesters of component C) are chosen from oligoesters comprising dicarboxylic acid units and diol units, preferably glycol, alkylglycol and/or polyalkylene polyglycol units, in particular obtained by polycondensation of one or more aromatic dicarboxylic acids or esters thereof with ethylene glycol and/or propylene glycol. These esters may optionally also comprise polyethylene glycol, polypropylene glycol, sulfoisophthalic acid, sulfobenzoic acid, isethionic acid, C₁-C₄-alcohols, oxyalkylated C₁-C₂₄-alcohols, oxyalkylated C₆-C₁₈-alkylphenols and/or oxyalkylated C₈-C₂₄-alkylamines as monomers.

Particular preference is given to the use of those oligoesters which are obtained by polycondensation of
a) 40 to 52 mol %, preferably 45 to 50 mol %, of one or more dicarboxylic acids or esters thereof,
b) 10 to 60 mol %, preferably 20 to 35 mol %, of ethylene glycol and/or propylene glycol,
c) 3 to 20 mol %, preferably 0 to 15 mol %, particularly preferably 10 to 15 mol %, of polyethylene glycol,
d) 0 to 10 mol % of a water-soluble addition product of from 5 to 80 mol of an alkylene oxide onto 1 mol of C₁-C₂₄-alcohols, C₆-C₁₈-alkylphenols or C₈-C₂₄-alkylamines and
e) 0 to 10 mol % of one or more polyols with 3 to 6 hydroxyl groups.

Suitable as component a) for the preparation of the copolyesters are, for example, terephthalic acid, phthalic acid, isophthalic acid, and the mono- and dialkyl esters with C₁-C₆-alcohols, such as dimethyl terephthalate, diethyl terephthalate and di-n-propyl terephthalate. Further examples of compounds which can be used as component a) for the preparation of the polyesters are oxalic acid, succinic acid, glutaric acid, adipic acid, fumaric acid, maleic acid, itaconic acid, and the mono- and dialkyl esters of the carboxylic acids with C₁-C₆-alcohols, e.g. diethyl oxalate, diethyl succinate, diethyl glutarate, methyl adipate, diethyl adipate, di-n-butyl adipate, ethyl fumarate and dimethyl maleate. If the suitable dicarboxylic acids can form anhydrides, the anhydrides of the carboxylic acids having at least 2 carboxyl groups are also suitable as compound of component a) for the preparation of the oligoesters, e.g. maleic anhydride, phthalic anhydride or succinic anhydride. As compound of component a), particular preference is given to using terephthalic acid, phthalic acid, isophthalic acid, and dimethyl, diethyl, dipropyl and dibutyl esters thereof. It is of course possible to use mixtures of different carboxylic acids or different esters. It is likewise possible to also use, for example, mixtures of carboxylic acids and ester or mixtures of carboxylic acids and anhydrides in the condensation.

As component c), polyethylene glycols with molar masses of from 500 to 5000, preferably from 1000 to 3000, are used.

Suitable as component d) for the preparation of the oligoesters are water-soluble addition products of from 5 to 80 mol of at least one alkylene oxide onto 1 mol of C₁-C₂₄-alcohols, C₆-C₁₈-alkylphenols or C₈-C₂₄-alkylamines. Preference is given to monomethyl ethers of polyethylene glycols. Alkylene oxides used for the preparation of the compounds of component d) are preferably ethylene oxide and mixtures of ethylene oxide and propylene oxide. Also suitable are mixtures of ethylene oxide together with propylene oxide and/or butylene oxide, mixtures of ethylene oxide, propylene oxide and isobutylene oxide or mixtures of ethylene oxide and at least one butylene oxide. These water-soluble addition products of the alkylene oxides are surfactants. If mixtures of alkylene oxides have been used for their preparation, then they can contain the alkylene oxides in blocks or else in random distribution.

Suitable alcohols which are alkoxylated are, for example, octyl alcohol, decyl alcohol, lauryl alcohol, myristyl alcohol or stearyl alcohol, but in particular methanol, and the alcohols having 8 to 24 carbon atoms obtainable by the Ziegler process, or the corresponding oxo alcohols. Of the alkylphenols, octylphenol, nonylphenol and dodecylphenol in particular are of importance for the preparation of the corresponding surfactants. Of the suitable alkylamines, use is made in particular of the C₁₂-C₁₈-monoalkylamines for the preparation of the water-soluble surfactants.

Suitable polyols of component e) are, for example, pentaerythritol, trimethylolethane, trimethylolpropane, 1,2,3-hexanetriol, sorbitol, mannitol and glycerol.

The synthesis of the oligoesters according to the invention takes place by processes known per se by heating the components a), b) and c) and optionally d) with the addition of a catalyst firstly at atmospheric pressure to temperatures of from 160 to about 220° C. The reaction is then continued under reduced pressure at temperatures of from 160 to about 240° C. while distilling off excess glycols. The known transesterification and condensation catalysts of the prior art are suitable for the reaction, such as, for example, titanium tetraisopropoxide, dibutyltin oxide or antimony trioxide/calcium acetate. For further details on carrying out the process, reference is made to EP 442 101.

Of particular suitability are also the polyesters known from EP 241 985 which, besides oxyethylene groups and terephthalic acid units, contain 1,2-propylene, 1,2-butylene and/or 3-methoxy-1,2-propylene groups, and glycerol units and are terminally capped with C₁-C₄-alkyl groups, the soil release polymers with a molar mass of from 900 to 9000 g/mol of ethylene terephthalate and polyethylene oxide terephthalate described in EP 253 567, where the polyethylene glycol units have molecular weights of from 300 to 3000 g/mol and the molar ratio of ethylene terephthalate to polyethylene oxide terephthalate is 0.6 to 0.95, and the polyesters with polypropylene terephthalate and polyoxyethylene terephthalate units which are terminally capped at least proportionally by C₁-C₄-alkyl or acyl radicals and known from EP 272 033.

Preference is likewise given to oligoesters of ethylene terephthalate and polyethylene oxide terephthalate in which the polyethylene glycol units have molecular weights of from 750 to 5000 g/mol and the molar ratio of ethylene terephthalate to polyethylene oxide terephthalate is 50:50 to 90:10 and whose use in detergents is described in German patent specification DE 28 57 292, and oligoesters with molecular weights of from 15 000 to 50 000 g/mol of ethylene terephthalate and polyethylene oxide terephthalate, where the polyethylene glycol units have molecular weights of from 1000 to 10 000 g/mol and the molar ratio of ethylene terephthalate to polyethylene oxide terephthalate is 2:1 to 6:1, which, according to DE 33 24 258, can be used in detergents.

Preference is likewise given to the oligoesters described in DE 19 644 034 of the formula 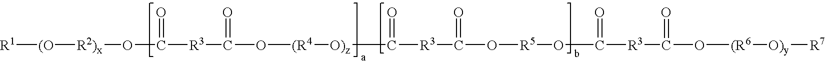
in which
R¹ and R⁷ are linear or branched C₁-C₁₈-alkyl,
R² and R⁶ are ethylene,
R³ is 1,4-phenylene,
R⁴ is ethylene,
R⁵ is ethylene, 1,2-propylene or random mixtures of any composition of the two,
x and y independently of one another, are a number between 1 and 500,
z is a number between 10 and 140,
a is a number between 1 and 12,
b is a number between 7 and 40,
where a+b is at least 11.

Preferably, independently of one another,
R¹ and R⁷ are linear or branched C₁-C₄-alkyl,
x and y are a number between 3 and 45,
z is a number between 18 and 70,
a is a number between 2 and 5,
b is a number between 8 and 12,
a+b is a number between 12 and 18 or between 25 and 35.

The oligoesters described in DE 19 644 034 are obtained from dimethyl terephthalate, ethylene glycol and/or propylene glycol, polyethylene glycol and C₁- to C₁₈-alkyl polyethylene glycol with the addition of a catalyst firstly by transesterification at temperatures of from 160 to about 220° C. and distillative removal of the methanol at atmospheric pressure and subsequent distillative removal of the excess glycols at temperatures of from 160 to about 240° C.

Preferably, the cosmetic formulations comprise 0.1 to 50% by weight, particularly preferably 0.5 to 10% by weight, especially preferably 1 to 4% by weight, of the polymers, based on the finished formulation.

The compounds of the formula (1) are preferably present in the cosmetic formulation in an amount of from 0.1 to 10% by weight, based on the finished cosmetic formulation.

The compounds of the formula (2) are preferably present in the cosmetic formulation in an amount of from 0.1 to 10% by weight, based on the finished cosmetic formulation.

The oligoesters of component C) are preferably present in the cosmetic formulation in an amount of from 0.1 to 10% by weight, based on the finished cosmetic formulation.

The cosmetic formulations are preferably shampoos, in particular hair shampoos, rinses, conditioners, cream rinses, gels, in particular hair gels, and other hair styling products.

Here, the total amount of surfactants, based on the finished cosmetic formulations, is preferably between 5 and 70% by weight, particularly preferably between 10 and 40% by weight, especially preferably between 12 and 35% by weight.

Preferred anionic surfactants are (C₁₀-C₂₀)-alkyl and alkylene carboxylates, alkyl ether carboxylates, fatty alcohol sulfates, fatty alcohol ether sulfates, alkylamide sulfates and sulfonates, fatty acid alkylamide polyglycol ether sulfates, alkane sulfates, alkanesulfonates and hydroxyalkanesulfonates, olefinsulfonates, acyl esters of isethionates, α-sulfo fatty acid esters, alkylbenzenesulfonates, alkylphenol glycol ether sulfonates, sulfosuccinates, sulfosuccinic monoesters and diesters, fatty alcohol ether phosphates, protein-fatty acid condensation products, alkyl monoglyceride sulfates and sulfonates, alkyl glyceride ether sulfonates, fatty acid methyltaurides, fatty acid sarcosinates, sulforicinoleates, amphoacetates or amphoglycinates and/or acylglutamates. The anionic surfactants are preferably used in the form of their water-soluble or water-dispersible salts, e.g. in the form of sodium, potassium, magnesium, ammonium, mono-, di- and triethanolammonium or alkylammonium salts.

The weight fraction of the anionic surfactants, based on the finished cosmetic formulations, is preferably 0.1 to 50% by weight, particularly preferably 7 to 30% by weight, especially particularly preferably 9 to 18% by weight.

Preferred cationic surfactants are quaternary ammonium salts, preferably di-(C₁₀-C₂₄)alkyldimethylammonium chloride and bromide, particularly preferably di-(C₁₂-C₁₈)alkyldimethylammonium chloride and bromide; (C₁₀-C₂₄)alkyldimethyl-ethylammonium chloride and bromide; (C₁₀-C₂₄)alkyltrimethylammonium chloride and bromide, preferably cetyltrimethylammonium chloride and bromide and (C₂₀-C₂₂)alkyltrimethylammonium chloride and bromide; (C₁₀-C₂₄)akyldimethylbenzylammonium chloride and bromide, preferably (C₁₂-C₁₈)alkyldimethylbenzylammonium chloride; N—(C₁₀-C₁₈)alkylpyridinium chloride and bromide, preferably N—(C₁₂-C₁₆)alkylpyridinium chloride and bromide; N—(C₁₀-C₁₈)alkylisoquinolinium chloride, bromide and monoalkyl sulfate; N—(C₁₂-C₁₈)alkylpolyoylaminoformylmethyl-pyridinium chloride; N—(C₁₂-C₁₈)alkyl-N-methylmorpholinium chloride, bromide and monoalkylsulfate; N—(C₁₂-C₁₈)alkyl-N-ethylmorpholinium chloride, bromide and monoalkyl sulfate; (C₁₆-C₁₈)alkylpentaoxethylammonium chloride; diisobutylphenoxy-ethoxyethyldimethylbenzylammonium chloride; salts of N,N-diethylaminoethylstearylamide and -oleylamide with hydrochloric acid, acetic acid, lactic acid, citric acid and phosphoric acid; N-acylaminoethyl-N,N-diethyl-N-methyl-ammonium chloride, bromide and monoalkyl sulfate; and/or N-acylaminoethyl-N,N-diethyl-N-benzylammonium chloride, bromide and monoalkyl sulfate, where the acyl radicals are preferably stearoyl or oleoyl radicals.

The weight fraction of the cationic surfactants, based on the finished cosmetic formulations, is preferably 1 to 10% by weight, particularly preferably 2 to 7% by weight and especially preferably 3 to 5% by weight.

Preferred nonionic surfactants are fatty alcohol ethoxylates (alkyl polyethylene glycols); alkylphenol polyethylene glycol; alkyl mercaptan polyethylene glycol; fatty amine ethoxylates (alkylaminopolyethylene glycols); fatty acid ethoxylates (acyl polyethylene glycols); polypropylene glycol ethoxylates (®Pluronics); fatty acid amide polyethylene glycols; N-alkyl-, N-alkoxypolyhydroxy fatty acid amide, preferably fatty acid N-methylglucamides and sucrose esters; polyglycol ethers; alkyl polyglycoside; and/or phosphoric esters (mono-, di- and triphosphoric esters ethoxylated and nonethoxylated).

The weight fraction of the nonionic surfactants, based on the finished cosmetic formulations, is preferably 1 to 20% by weight, particularly preferably 2 to 10% by weight, especially preferably 3 to 7% by weight.

Preferred amphoteric surfactants are N—(C₁₂-C₁₈)alkyl-β-aminopropionate and N—(C₁₂-C₁₈)alkyl-β-iminodipropionate as alkali metal and mono-, di- and trialkylammonium salts; N-acylaminoalkyl-N,N-dimethylacetobetaine, preferably
N—(C₈-C₁₈)acylaminopropyl-N,N-dimethylacetobetaine; (C₁₂-C₁₈)alkyldimethyl-sulfopropylbetaine; amphoteric surfactants based on imidazoline (trade names Miranol®, Steinapon®), preferably the sodium salts of 1-(β-carboxymethyloxyethyl)-1-(carboxymethyl)-2-laurylimidazolinium; amine oxides, e.g.
(C₁₂-C₁₈)alkyldimethylamine oxide; and/or fatty acid amidoalkyldimethylamine oxides.

The weight fraction of the amphoteric surfactants, based on the finished cosmetic formulations, is preferably 0.5 to 20% by weight and particularly preferably 1 to 10% by weight.

Furthermore, foam-boosting cosurfactants from the group of alkylbetaines, alkylamidobetaines, aminopropionates, aminoglycinates, imidazoliniumbetaines, sulfobetaines, amine oxides, fatty acid alkanolamides and polyhydroxyamides can be used in the cosmetic formulations.

Particularly preferred surfactants are lauryl sulfate, laureth sulfate, cocoamidipropylbetaine, sodium cocoyl glutamate, disodium laureth sulfosuccinate and/or coconut fatty acid diethanolamide.

In addition, the cosmetic formulations can comprise superfatting agents, fats, waxes, stabilizers, biogenic active ingredients, glycerol, preservatives, pearlizing agents, dyes and fragrances, solvents, opacifiers, thickeners, dispersants, protein derivatives (e.g. gelatin), collagen hydrolyzates, natural- and synthetic-based polypeptides, egg yolk, lecithin, lanolin, lanolin derivatives, fatty alcohols, silicones, deodorizing agents, substances with a keratolytic and keratoplastic effect, enzymes, carrier substances, moisturizing substances and/or antimicrobial agents.

Preferred superfatting agents are polyethoxylated lanolin derivatives, lecithin derivatives, polyol fatty acid esters, monoglycerides and fatty acid alkanolamides, the latter being suitable as foam stabilizers at the same time.

Preferred fats are glycerides, and suitable waxes are, inter alia, beeswax, paraffin wax or microcrystalline waxes, optionally in combination with hydrophilic waxes, e.g. cetylstearyl alcohol.

Stabilizers which can be used are metal salts of fatty acids, such as, for example, magnesium stearate, aluminum stearate and/or zinc stearate.

Biogenic active ingredients are understood as meaning, for example, plant extracts and vitamin complexes.

Suitable preservatives are, for example, phenoxyethanol, formaldehyde donors, parabens, pentanediol and sorbic acid.

Dyes which can be used are the substances approved and suitable for cosmetic purposes.

Preferred thickeners and dispersants are sodium chloride, potassium chloride, ammonium chloride, sodium sulfate, fatty acid alkylolamides, cellulose derivates, for example hydroxyethylcellulose, guar gum, polyvinyl alcohol, polyvinylpyrrolidone, hydroxypropyl guar gum, starch and starch derivatives, and natural gums, carboxyvinyl polymers (e.g. the Carbopol® grades 934, 940, 941, 956, 980, 981, 1342 and 1382). Particularly suitable thickeners and dispersants are ethylene glycol esters of fatty acids having 14 to 22, particularly preferably 16 to 22, carbon atoms, in particular mono- and diethylene glycol stearate. Preference is also given to stearin monoethanolamide, stearin diethanolamide, stearin isopropanolamide, stearin monoethanolamide stearate, stearyl stearate, cetyl palmitate, glyceryl stearate, stearamide diethanolamide distearate, stearamide monoethanolamide stearate,

N,N-dihydrocarbyl-(C₁₂-C₂₂)-, preferably (C₁₆-C₁₈), amidobenzoic acid and soluble salts thereof, and/or N,N-di(C₁₆-C₁₈)amidobenzoic acid and derivatives thereof.

Based on the finished cosmetic formulations, the thickeners and dispersants are used preferably in an amount of from 0.5 to 10% by weight, particularly preferably in an amount of from 0.5 to 5% by weight and especially preferably in an amount of from 1 to 4% by weight.

The desired viscosity of the cosmetic formulations can be established by adding water and/or organic solvents or by adding a combination of organic solvents and thickeners.

In principle, suitable organic solvents are all mono- or polyhydric alcohols and ethoxylated alcohols. Preference is given to alcohols having 1 to 4 carbon atoms, such as ethanol, propanol, isopropanol, n-butanol and isobutanol, glyceryl and mixtures of said alcohols. Further suitable solvents are, for example, triacetin (glycerol triacetate) and 1-methoxy-2-propanol.

Based on the finished formulations, the cosmetic formulations comprise the alcohols preferably in amounts by weight of from 0.1 to 50% by weight.

Suitable carrier materials are preferably vegetable oils, natural and hydrogenated oils, waxes, fats, water, alcohols, polyols, glycerol, glycerides, liquid paraffins, liquid fatty alcohols, sterol, cellulose and cellulose derivates.

Fungicidal active ingredients which may be used are ketoconazole, oxiconazole, terbinafine, bifonazole, butoconazole, cloconazole, clotrimazole, econazole, enilconazole, fenticonazole, isoconazole, miconazole, sulconazole, tioconazole, fluconazole, itraconazole, terconazole, naftifine, zinc pyrethione and octopirox.

Further suitable care substances are allantoin and bisabolol, preferably in amounts by weight of from 0.0001 to 10% by weight.

Preferred cationic polymers are cationic cellulose derivatives, cationic starch, copolymers of diallylammonium salts and arylamides, quaternized vinylpyrrolidone/vinylimidazole polymers, condensation products of polyglycols and amines, quaternized coliagen polypeptides, quaternized wheat polypeptides, polyethyleneimine, cationic silicone polymers, such as, for example amidomethicones, copolymers of adipic acid and dimethylaminohydroxypropyldiethylenetriamine, polyaminopolyamide and cationic chitin derivatives, such as, for example, chitosan.

Suitable silicone compounds are, for example, dimethylpolysiloxane, methylphenyl-polysiloxanes, cyclic silicones, and amino-, fatty acid-, alcohol-, polyether-, epoxy-, fluorine- and/or alkyl-modified silicone compounds, and polyalkylsiloxanes, polyalkylarylsiloxanes, polyethersiloxane copolymers, as described in U.S. Pat. No. 5,104,645 and the specification cited therein, which, at room temperature, may be present either in liquid form or else in the form of a resin.

The cosmetic formulations can be mixed with conventional ceramides, pseudoceramides, fatty acid N-alkylpolyhydroxyalkylamides, cholesterol, cholesterol fatty acid esters, fatty acids, triglycerides, cerebrosides, phospholipids and similar substances.

Suitable pearlescence-imparting compounds are fatty acid monoalkanolamides; fatty acid dialkanolamides; monoesters and diesters of alkylene glycol, in particular those of ethylene glycol, propylene glycol or oligomers thereof and higher fatty acids, e.g. palmitic acid, stearic acid, behenic acid or mixtures thereof; monoesters or diesters of alkylene glycols with fatty acids; fatty acids and metal salts thereof; monoesters or polyesters of glycerol with carboxylic acids; and/or ketosulfones. Particularly preferred pearlescence-imparting components are ethylene glycol distearate and polyethylene glycol distearate with 3 glycol units.

Preferred moisturizing substances are isopropyl palmitate, glycerol and/or sorbitol. Based on the finished formulations, they are preferably present in amounts by weight of from 0.1 to 50% by weight.

The examples below are intended to illustrate the subject-matter of the invention in more detail without limiting it thereto.

### EXAMPLES

Tresses of bleached blonde hair were each colored with 2 ml for 30 minutes using a standard commercial color (Wella Viva Pure red). L and E values in the CILAB system were determined using a spectrophotometer (Datacolor Mercury 2000). Here, the L values give a measure of the lightness and the E values a measure of the shade. The tresses were then washed four times for one minute in each case with 0.5 ml of standard or test shampoo and rinsed in each case for 30 seconds. L and E values were then measured and the differences determined as DL and DE.

| | Test formulations | | | | |
|---|---|---|---|---|---|
| Formulation: Standard | S | A | B | C | D |
| Sodium laureth sulfate | 48.1 | 48.1 | 48.1 | 48.1 | 48.1 |
| (27% active) | | | | | |
| Cocamidopropylbetaine | 6.7 | 6.7 | 6.7 | 6.7 | 6.7 |
| (30% active) | | | | | |
| Polyglycol 35000 S | — | 1.0 | — | — | 0.3 |
| Genamin KSL | — | — | 1.0 | — | 0.3 |
| Aristoflex PEA | — | — | — | 1.0 | 0.3 |
| Demin. water | ad 100 | ad 100 | ad 100 | ad 100 | ad 100 |
| NaCl | 1.8 | 1.8 | 1.8 | 1.8 | 1.8 |

Results of the color measurements (data in units of reflection):

| | S | A | B | C | D |
|---|---|---|---|---|---|
| DL | 4.0 | 1.2 | 2.5 | 1.5 | 1.1 |
| DE | 7.5 | 3.5 | 3.5 | 4.1 | 3.1 |

Significant effectiveness of the individual active ingredients and also of the combination of all three active ingredients can be seen.

### Formulation Example 1

### Hair Shampoo

| Components | | Amount in % by wt. |
|---|---|---|
| 1 | Genapol LRO liquid | 11.1 |
| 2 | Fragrance | 0.3 |
| 3 | Demin. water | ad 100 |
| 4 | Genagen KB | 12.0 |
| 5 | Genagen LAA | 11.6 |
| 6 | Polyglycol 35000 S | 1.0 |
| 7 | Aristoflex PEA | 1.0 |
| 8 | Citric acid (50% demin. water) | 1.2 |
| 9 | Dye solution | q.s. |
| 10 | Preservative | q.s. |

Preparation:

Component 1 was initially introduced, then the other components were stirred in in the order given.

### Formulation Example 2

### Cream Rinse

| Components | | Amount in % by weight |
|---|---|---|
| 1 | Genamin DSAC | 1.5 |
| 2 | Hostacerin T-3 | 1.5 |
| 3 | Cetyl alcohol | 2.5 |
| 4 | Paraffin oil, high-viscosity | 1.0 |
| 5 | Genamin KSL | 2.0 |
| 6 | Demin. water | ad 100 |
| 7 | Polyglycol 20000 S | 1.0 |
| 8 | Preservative | q.s. |
| 9 | Perfume oil | 0.3 |
| 10 | Dye solution | q.s. |
| 11 | Citric acid | q.s. |

Preparation:

Components 1 to 4 were melted at about 75° C. Components 5 to 8 were then heated to about 75° C. and added with stirring. The mixture was then stirred until cold. At about 35° C., components 9 and 10 were stirred in and, finally, the pH was adjusted to pH 4 with citric acid.

List of Products Used:

| | |
|---|---|
| Polyglycol 20000 S (Clariant GmbH) | PEG-450, polyethylene glycol |
| | with an average molar mass of |
| | 20 000 g/mol |
| Polyglycol 35000 S (Clariant GmbH) | PEG-800, polyethylene glycol |
| | with an average molar mass of |
| | 35 000 g/mol |
| Genagen ® LAA (Clariant GmbH) | Lauryl amphoacetate, Na salt |
| Genagen ® KB (Clariant GmbH) | Cocoylbetaine |
| Genapol ® LRO (Clariant GmbH) | Sodium laureth sulfate |
| Genamin ® KSL (Clariant GmbH) | PEG-5 stearyl ammonium lactate |
| Hostacerin ® T-3 (Clariant GmbH) | Ceteareth-3 |
| Genamin ® DSAC (Clariant GmbH) | Distearyldimonium chloride |
| Aristoflex ® PEA (Clariant GmbH) | Polypropylene terephthalate |

1. A method for preserving color in keratin fibers, said method comprising contacting said keratin fibers with a cosmetic formulation comprising one or more polymers selected from the group consisting of:
A) polyethylene glycols of the formula (1)
H(OCH₂CH₂)ₙOH (1),
in which n is an integer between 150 and 900,
B) ethoxylated quaternary ammonium compounds of the formula (2) 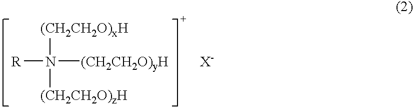
in which
R is C₄- to C₃₀-alkyl,
x, y and z, in each case independently of one another, are a number from 1 to 300, and
X⁻ is an anionic counterion, and
C) oligoesters which are obtained by condensation of one or more dicarboxylic acids or esters thereof and one or more polyhydric alcohols, and mixtures thereof.

2. The method of claim 1, wherein the keratin fibers are human hair.

3. The method of claim 1, wherein the polymers are present in a cosmetic formulation.

4. The method of claim 1, wherein n in formula (1) is an integer between 220 and 800.

5. The method of claim 1, wherein the cosmetic formulation comprises 0.1 to 10% by weight of the compounds of the formula (1), based on the cosmetic formulation.

6. The method of claim 1, wherein R in formula (2) is C₈- to C₂₂-alkyl and the sum x+y+z is a number from 4 to 50.

7. The method of claim 1, wherein the anionic counterion X⁻ in formula (2) is selected from the group consisting of halide anions, OH⁻, a deprotonated weak acid, and mixtures thereof.

8. The method of claim 1, wherein the cosmetic formulation comprises 0.1 to 10% by weight of the compounds of the formula (2), based on the cosmetic formulation.

9. The method of claim 1, wherein the oligoesters of component C) are chosen from compounds which are obtained by polycondensation of
a) 40 to 52 mol % of one or more aromatic dicarboxylic acids or esters thereof,
b) 10 to 60 mol % of ethylene glycol or propylene glycol or mixtures thereof,
c) 0 to 20 mol % of polyethylene glycol,
d) 0 to 10 mol % of a water-soluble addition product of from 5 to 80 mol of an alkylene oxide onto 1 mol of a compound selected from the group consisting of C₁-C₂₄-alcohols, C₆-C₁₈-alkylphenols C₈-C₂₄-alkylamines, and mixtures thereof, and
e) 0 to 10 mol % of one or more polyols with 3 to 6 hydroxyl groups.

10. The method of claim 1, wherein the cosmetic formulation comprises 0.1 to 10% by weight of oligoesters of component C), based on the cosmetic formulation.

11. The method of claim 1, wherein the cosmetic formulation comprises one or more polyethylene glycols of the formula (1) as in component A) and one or more ethoxylated quaternary ammonium compounds of the formula (2) as in component B).

12. The method of claim 1, wherein the cosmetic formulation comprises one or more polyethylene glycols of the formula (1) as in component A) and one or more oligoesters as in component C).

13. The method of claim 1, wherein the cosmetic formulation comprises one or more ethoxylated quaternary ammonium compounds of the formula (2) as in component B) and one or more oligoesters as in component C).

14. The method of claim 1, wherein the cosmetic formulation comprises one or more polyethylene glycols of the formula (1) as in component A), one or more ethoxylated quaternary ammonium compounds of the formula (2) as in component B) and one or more oligoesters as in component C).

15. A cosmetic formulation comprising at least two polymers chosen from
A) polyethylene glycols of the formula (1)
H(OCH₂CH₂)ₙOH (1),
in which n is an integer between 150 and 900,
B) ethoxylated quaternary ammonium compounds of the formula (2) 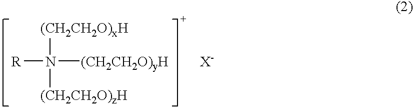
in which
R is C₄- to C₃₀-alkyl,
x, y and z, in each case independently of one another, are a number from 1 to 300, and
X⁻ is an anionic counterion, and
C) oligoesters which are obtained by condensation of one or more dicarboxylic acids or esters thereof and one or more polyhydric alcohols.

16. The cosmetic formulation as claimed in claim 15, which comprises one or more polyethylene glycols of the formula (1) as in component A) and one or more ethoxylated quaternary ammonium compounds of the formula (2) as in component B).

17. The cosmetic formulation as claimed in claim 15, which comprises one or more polyethylene glycols of the formula (1) as in component A) and one or more oligoesters as in component C).

18. The cosmetic formulation as claimed in claim 15, which comprises one or more ethoxylated quaternary ammonium compounds of the formula (2) as in component B) and one or more oligoesters as in component C).

19. The cosmetic formulation as claimed in claim 15, which comprises one or more polyethylene glycols of the formula (1) as in component A), one or more ethoxylated quaternary ammonium compounds of the formula (2) as in component B) and one or more oligoesters as in component C).

20. The method of claim 1, wherein the oligoesters of component C) are obtained by condensation of one or more dicarboxylic acids or esters thereof and one or more polyhydric alcohols in the presence of an addition product of an alkylene oxide onto a compound selected from the group consisting of an alcohol, an alkylphenol, and alkylamine, and mixtures thereof.

21. The method of claim 20, wherein the addition product is methyl polyethylene glycol.

22. The method of claim 7, wherein the deprotonated weak acid is deprotonated lactic acid.

23. The cosmetic formulation of claim 15, wherein the oligoesters of component C) are obtained by condensation of one or more dicarboxylic acids or esters thereof and one or more polyhydric alcohols in the presence of an addition product of an alkylene oxide onto a compound selected from the group consisting of an alcohol, an alkylphenol, and alkylamine, and mixtures thereof.

24. The cosmetic formulation of claim 23, wherein the addition product is methyl polyethylene glycol.

25. The method of claim 9, wherein component a) is 45 to 50 mol % of one or more aromatic dicarboxylic acids or esters thereof.

26. The method of claim 9, wherein component b) is 20 to 35 mol % of one or more of ethylene glycol or propylene glycol or mixtures thereof.

27. The method of claim 9, wherein component c) is 0 to 15 mol % of polyethylene glycol.

28. The method of claim 9, wherein component c) is 10 to 15 mol % of polyethylene glycol.
